SEQUENCE LISTING (18 sequences)

SEQ ID NO: 1 - DNA, 20 bp, Bos taurus
ataagcccgacctgccgacc

SEQ ID NO: 2 - DNA, 20 bp, Bos taurus
ggaagtgttgcgtttagcgg

SEQ ID NO: 3 - DNA, 20 bp, Bos taurus
gccaccgtagcccctcatag

SEQ ID NO: 4 - DNA, 20 bp, Bos taurus
ttccgcgcgaaatgcttcca

SEQ ID NO: 5 - DNA, 22 bp, Artificial Sequence
  Synthetic Oligonucleotide
caaggtctaagtcatcttcccc

SEQ ID NO: 6 - DNA, 20 bp, Artificial Sequence
  Synthetic Oligonucleotide
ggacactcgaaatcccatcc

SEQ ID NO: 7 - DNA, 22 bp, Artificial Sequence
  Synthetic Oligonucleotide
caaggtctaagtcatcttcccc

SEQ ID NO: 8 - DNA, 19 bp, Artificial Sequence
  Synthetic Oligonucleotide
ctttgcggagggaccaatg

SEQ ID NO: 9 - DNA, 20 bp, Artificial Sequence
  Synthetic Oligonucleotide
gggcagagctagagtctcac

SEQ ID NO: 10 - DNA, 19 bp, Artificial Sequence
  Synthetic Oligonucleotide
ctttgcggagggaccaatg

SEQ ID NO: 11 - DNA, 20 bp, Artificial Sequence
  Synthetic Oligonucleotide
agagcaatcgtccagtcatg

SEQ ID NO: 12 - DNA, 20 bp, Artificial Sequence
  Synthetic Oligonucleotide
gtggacaggaacatcaggag

SEQ ID NO: 13 - DNA, 25 bp, Artificial Sequence
  source 1..23: synthetic oligonucleotide
  misc_feature 10: /note=“integrated ZEN quencher, used in the probe for the qPCR (available from Integrated DNA Technologies Inc., Coralville, Iowa, USA)”
cgctccaggnaagacatccctcttg

SEQ ID NO: 14 - DNA, 21 bp, Artificial Sequence
  Synthetic Oligonucleotide
tgaccctgatagaccttcctg

SEQ ID NO: 15 - DNA, 21 bp, Artificial Sequence
  Synthetic Oligonucleotide
gttgatggtctcctgcctaag

SEQ ID NO: 16 - DNA, 23 bp, Artificial Sequence
  Synthetic Oligonucleotide
  misc_feature 10: /note=“grated ZEN quencher, used in the probe for the qPCR (available from Integrated DNA Technologies Inc., Coralville, Iowa, USA)”
caaccagccnacccgacattgaa

SEQ ID NO: 17 - protein, 91 aa, Artificial Sequence
  IRF3 frameshift mutation
MGTQKPRILPWLISQLDRGELEGVAWLGESRTRFRIPWKHGLRQDAQQEDFGIFQAWAVASGAYTPGKDKPDLPRRTTARTPKTHTKSMSL

SEQ ID NO: 18 - protein, 26 aa, Artificial Sequence
  IRF7 frameshift mutation
MAEAPDRGTPRVLFGDWLLGEVSSGR

(19) United States
(12) Patent Application Publication
(10) Pub. No.: US 2022/0315953 A1
(43) Pub. Date: Oct. 6, 2022

(54) MDBK IRF3/IRF7 KNOCK OUT MUTANT CELL AND ITS USE FOR VACCINE PRODUCTION

(71) Applicant: Intervet Inc., Madison, NJ (US)

(72) Inventors: Alexander Martijin Langereis, Gennep (NL); Ad De Groof, Groesbeek (NL); Willem Bartjan Simmelink, Nijmegen (NL); Paul Vermeij, St. Anthonis (NL)

(73) Assignee: Intervet Inc., Madison, NJ (US)

(21) Appl. No.: 17/615,961

(22) PCT Filed: Jun. 5, 2020
(86) PCT No.: PCT/EP2020/065625

§ 371 Date: Dec. 2, 2021

(30) Foreign Application Priority Data
Jun. 7, 2019 (EP) 19178949.4

(51) Int. Cl. C12N 15/90; C12N 5/071; C07K 14/47; C12N 7/00

(52) U.S. Cl. CPC C12N 15/907; C12N 5/0686; C07K 14/4702; C12N 7/00; C12N 2510/00; C12N 2760/18521; C12N 2760/18534; C12N 2760/18551

(57) ABSTRACT

The present invention pertains to a Madin-Darby bovine kidney (MDBK) cell, wherein the interferon regulatory factors (IRF) IRF3 and/or IRF7 encoding genes are functionally inactivated. The invention also pertains to a cell culture comprising the MDBK cell, use of the MDBK cell culture, a method for the production of a virus using the cell and a vaccine prepared by using the cell.

The present invention pertains to a Madin-Darby bovine kidney (MDBK) cell, wherein the interferon regulatory factors (IRF) IRF3 and/or IRF7 encoding genes are functionally inactivated, i.e. “knocked out”. In other embodiments, the present invention pertains to a cell culture comprising the MDBK IRF3 and/or IRF7 knockout mutant cell, use of the cell culture for virus production, to a method for the preparation of a virus and to a vaccine composition comprising the cell culture or the virus produced from the cell culture. In still another embodiment, the present invention pertains to a method of CRISPR-Cas9 mediated gene editing for performing gene knockout of the IRF3 and/or IRF7 encoding genes.

### GENERAL BACKGROUND

The propagation of viruses for the purpose of vaccine production requires the availability of susceptible host cells. Usually, depending on the virus species and the type of host cell used, these host cells will be grown in cell culture. Therefore, the production of virus vaccines requires establishing cell lines for growth and replication of viruses.

Vaccines developed from viruses grown in mammalian cell cultures are of major importance in both human and animal health. The economic and sociological losses due to viral infections in human populations and the animal-production industry are significant, and necessitate the use of large-scale vaccination programs. Therefore, efforts are continually underway to improve cell culture systems that enhance the ability to increase the effectiveness of vaccine production and make the process more economical.

For many viruses, such as bovine respiratory syncytial virus (BRSV), a possibility of studying and producing the virus are bovine kidney cells, such as Madin-Darby bovine kidney (MDBK) cells, a cell line commonly used for vaccine production. The MDBK cell line was originally derived from a kidney of an apparently normal adult steer in February 18, 195, by S. H. Madin and N .B. Darby (Madin S H, Darby N B Jr., Proc. Soc. Exp. Biol. Med., Vol. 98, p. 574-576, 1958).

BRSV is a single stranded RNA virus classified as a pneumovirus in the Paramyxovirus family. BRSV infections associated with lower respiratory tract infections that occur predominantly in young beef and dairy cattle. Passively derived maternal immunity does not appear to prevent BRSV infections but reduces the severity of disease. Initial exposures to the virus are associated with severe respiratory disease in susceptible animals; subsequent exposures result in mild to subclinical disease. BRSV is an important virus in the bovine respiratory disease complex because of its frequency of occurrence, predilection for the lower respiratory tract, and ability to predispose the respiratory tract to secondary bacterial infection. In outbreaks, morbidity tends to be high, and the case fatality rate can be 0-20%, thus causing huge economic loss. Inactivated and modified-live vaccines are available (e.g. Bovilis® Bovipast® RSP or Vista 5 SQ both from MSD Animal Health) and may serve to reduce losses associated with BRSV.

Current vaccine production systems using mammalian cell culture only give limited yields and a relatively low concentration of virus. As a consequence, a concentration step is often necessary, which complicates the production process. Higher yields in cell culture would be desirable to enable larger doses and combinations with other vaccines.

Therefore, it is desirous to increase viral yields and titers of current production systems using cell cultures. However, increasing viral yields and titers in cell cultures may be hampered by the cells innate immune system, which constitutes an effective defense against viral infections.

The innate immune system constitutes the first line of host defense during infection. Upon viral infections, the cell's innate immune system is stimulated after recognition of for example pathogen-associated molecular patterns (PAMPs), which results in activation of antiviral response genes and the induction of a cellular anti-viral state. A fundamental part of the cell's innate immune system is the induction of type I interferon (IFN) expression in response to viral infections. Type I interferon expression leads to the initiation of antiviral signaling cascades in infected cells as well as in neighboring non-infected cells, resulting in an antiviral response capable of controlling or partially controlling most viral infections.

A part of the interferon signaling cascade is the activation of transcription factors such as NF-KB and IRF3 and/or IRF7. These transcription factors lead to the activation of the IFN-α/β promoters resulting in IFN-α/β transcription and expression. IFN-α/β is secreted from infected host cells and this results in the induction of an antiviral state in neighboring non-infected cells. An important aspect adding to the complexity of IFN production and regulation is the existence of a positive feedback loop, which represents a way to significantly enhance the IFN response. Whereas IRF3 is constitutively expressed, IRF7 expression is weak in unstimulated cells, but is dramatically upregulated in response to virus infection (T. H. Mogensen, Clinical Microbiology Reviews, Vol. 22(2), 2009, p. 240-273).

Activation of the type I IFN response also results in a variety of complementary antiviral effects on the cell. For example, activation of the IFN induction cascade through IRF3 can lead to the induction of certain IFN-stimulated genes (ISGs), which have an antiviral activity in the absence of IFN. IRF3 possess a distinct activity that induces apoptosis in virus infected cells (Randall, R. E. and S. Goodbourn, The Journal of general virology, 2008. 89 (Pt 1), p. 1-47, and Chattopadhyay, S. and G. C. Sen, Cell cycle, 2010. 9(13), p. 2479-80).

The type I IFN signaling cascade constitutes an extremely powerful antiviral response that is capable of controlling most if not all virus infections in the absence of adaptive immunity. However, it rarely works to full capacity because almost all viruses have evolved type I IFN antagonists that use a wide variety of mechanisms to circumvent the type I IFN response by either directly or indirectly targeting the type I IFN-induction or type I IFN-signaling cascades, or both.

Several attempts were made in the art to increase viral growth rates by counteracting the type I IFN system. For example, counteracting the type I IFN induction/signaling cascades by using viral IFN antagonists or inhibitors may have beneficial effects on viral growth by counteracting the cell's complementary antiviral mechanisms.

One of the most successful approaches to producing virus vaccines has been the generation of attenuated viruses, which are administered to mimic natural infection and induce protective immunity without causing disease. One general approach to producing attenuated viruses would be to engineer viruses so as to disable their capacity to circumvent the type I IFN response. This is feasible because viral anti-IFN proteins are usually dispensable for virus replication in cell culture, and viruses in which the genes encoding these proteins have been knocked out are attenuated in vivo. However, one problem which arises from knocking out viral type I IFN resistance genes is that it can be difficult to grow such viruses to high titer in tissue culture cells which produce and respond to type IFN. Normally, IFN-sensitive viruses are grown in cells that have lost the ability to produce type I IFN due to spontaneous gene deletions (Desmyter J, Melnick J L, Rawls W E, J Virol. 1968; 2(10), p. 955-61; Mosca J D, Pitha P M Mol Cell Biol. 1986; 6(6), p. 2279-83).

D. F. Young et al., J Virol. 2003; 77(3), p. 2174-2181, demonstrate how cells, including MRCS cells (which are human diploid cells suitable for the production of attenuated viruses for use in humans), can be engineered to no longer respond to IFN. It is demonstrated that RSVs from which the NS1, NS2, SH, or glycoprotein G gene has been deleted can be grown to much higher titers in these IFN-nonresponsive cells than in the parental cells.

However, genetically engineering cell lines is considered time consuming and their use may create regulatory problems for vaccine manufacturers. Therefore, WO 2014/199166 A1 aims to overcome the problem of increasing viral growth rates and overall viral titer in cells by providing a method comprising adding an inhibitor of the IFN induction and/or signaling cascade to the cells in which the virus is cultured. These inhibitors may target one or more components of the IFN induction and/or signaling cascade, such as TLR3, MDA5, RIG-I, Cardif, TBK1/IKK₂₄₉ , IKKα/β, IRF3, NFKB or ATF-2/C-JUN. The inhibitors include small molecule inhibitors, such as siRNA, miRNA, lipocalins, as well as antibodies or antibody fragments.

Another approach to improving proliferation efficiency is suggested in JP 2013-236622 A. Therein, a method for the production of influenza viruses is provided, the method comprising infecting Madin- Darby canine kidney (MDCK) cells with influenza viruses and incubating the cell culture composition, wherein the MDCK cells have enhanced propagation efficiency of influenza virus by suppressing the expression and/or function of IRF7 or mitochondrial antiviral signaling protein (MAVS). The suppression of the expression of IRF7 and MAVS is attained by introducing siRNA, which is short- chain double-stranded RNA that degrades the mRNA of the target gene and suppresses its expression, and which is designed based on the sequence of the mRNA of the target genes.

However, the approaches suggested in the art of adding inhibitors or antagonists to inactivate one or more targets of the interferon signaling cascade have the disadvantages that they inhibit these targets only transiently and do not lead to a permanent inactivation of the type I IFN system. Moreover, the inhibitors or antagonists added to the cell culture may still be present in the vaccine product. Therefore, the presence of inhibitors may lead to unwanted side effects in the vaccinated subject and may also require regulatory review and safety assessment.

Therefore, there is a need in the art to increase viral growth rates and overall viral titer (yields) in cell cultures. In particular, there is a need in the art to increase yields of virus production in cell culture for use of the virus in the production of vaccines.

### OBJECT OF THE INVENTION

It is thus an object of the present invention to provide a virus production system which overcomes one or more of the prior art problems related to the addition of external type I IFN inhibitors. In particular, it is an object of the present invention to provide a virus production system which leads to increased viral yields. Further, it is an object of the present invention to stably and permanently inactivate the innate immune system of a cell used for virus production.

In the present invention, it has now been found that a cell's innate immune system can be stably and permanently inactivated without the addition of external inhibitors by functional inactivation (referred to herewith as “knock out”) of the IRF3 and/or IRF7 genes. The resulting knock out mutant cell line can beneficially be used for virus production with higher viral yields compared to the wild type cell parental cell line. Surprisingly, the viral yields of an engineered MDBK IRF3/IRF7 deficient single cell clone were remarkably higher than the yields from a different MDBK cell line that has spontaneously lost the ability to evoke a type I interferon response upon viral infection.

### SUMMARY OF THE INVENTION

The present invention provides an MDBK cell, wherein the interferon regulatory factors IRF3 and/or IRF7 encoding genes are functionally inactivated, i.e. “knocked out”. This cell is also referred to in the following as “MDBK ΔIRF3 and/or ΔIRF7 knock out mutant cell”.

Further, a cell culture comprising the cell of the present invention is provided. In another embodiment, the cell is virus infected and thus is particularly suitable for virus production, such as for production of BRSV.

In another embodiment, there is provided a method for the production of a virus, the method comprising:
(a) culturing the cell according to the present invention,
(b) infecting the cell with a virus,
(c) allowing the virus to replicate, and
(d) optionally isolating the virus from the cell culture.

In still another embodiment, there is provided a vaccine composition comprising the cell culture of the present invention or a virus isolated therefrom and a pharmaceutically acceptable carrier.

In still another embodiment, there is provided a method for the preparation of a vaccine composition comprising a virus, the method comprising:
(a) culturing the cell according to the present invention,
(b) infecting the cell with a virus,
(c) allowing the virus to replicate,
(d) optionally isolating the virus from the cell culture, and
(e) mixing the virus or the cell culture infected with the virus with a pharmaceutically acceptable carrier.

In a particularly preferred embodiment, the pharmaceutically acceptable carrier of the vaccine composition comprises a natural deep-eutectic solvent (NADES) having a water activity of less than about 0.8.

In still another embodiment, there is provided a method of CRISPR-Cas9 mediated gene editing, characterized in that the method comprises the steps of:
(a) providing an MDBK cell and
(b) performing functional gene inactivation of IRF3 and/or IRF7 encoding genes of the

MDBK cell.

### DETAILED DESCRIPTION OF THE INVENTION

It has been found that a genetically engineered MDBK cell in which the IRF3 and/or IRF7 genes are functionally inactivated show a disrupted interferon I pathway signaling. Since the interferon I pathway signaling is a central part of the cellular defense against virus infection, the disrupted interferon I signaling results in a deficit in the cell's antiviral response as part of the cell's innate immune system. This prevents forming an anti-viral state in uninfected cells. Therefore, a genetically engineered cell in which the IRF3 and/or IRF7 genes are functionally inactivated is particularly suitable for virus production. Hence, a virus produced in this genetically engineered cell can be obtained in higher yields compared to the parental cell with a functional innate immune system, and thus the cell can be beneficially used for the production of a virus vaccine.

The cell used for IRF3 and/or IRF7 gene knockout is a Madin-Darby bovine kidney (MDBK) cell. The MDBK cell line is well established for virus production and was originally derived from a kidney of an apparently normal adult steer in 1957 by S. H. Madin and N. B. Darby. MDBK cell lines are commercially available, for example, via the American Type Culture Collection (ATCC) or the European Collection of Authenticated Cell Cultures (ECACC). A commercially available MDBK cell line is MDBK ATCC® CCL 22 (ECACC 90050801). A specific lot of CCL-22, MDBK (NBL-1) bovine kidney cells was found to be positive for bovine viral diarrhea virus (BVDV), following an investigation by ATCC (December 2015; https://www.atcc.org/products/all/CCL-22.aspx#characteristics). Nowadays, cell lines batches are routinely tested for presence of extraneous agents to confirm that they are free of BVDV, and thus can safely be used for virus production.

IRF3 and IRF7 are present as homo and heterodimers in cells and show functional redundancy. Therefore, in order to effectively disrupt the interferon pathway signaling resulting in abolishing the cell's antiviral response, it is preferred to functionally inactive both genes simultaneously (also referred to herewith as “MDBK ΔIRF3/IRF7 double knock out mutant cell”). In the present invention, it has surprisingly been found that a cell line originating from a ΔIRF3 and/or IRF7 double knock out mutant cell can be cultured and infected with BRSV virus, with resulting titers that are higher than those obtained from a MDBK cell line that spontaneously lost the capacity of a functional type I interferon response. This is a surprise since it was expected that the latter cell line, having completely lost its type I interferon response, would be the best option for growing a virus, or at least as good as any specific knock out mutant.

The term “knock out” or “gene knock out” as used in the present invention relates to any kind of gene editing technology in which the functional expression of a target gene (i.e. a functional protein) is inhibited, i.e. functionally inactivated, or in which a non-functional (truncation)mutant of the target gene (i.e. the protein) is created. In the present invention, the target genes are the interferon regulatory factors IRF3 and IRF7 encoding genes. Therefore, gene knock out can be achieved, for example, by disruption of a cell's genome, such as by incorporation of a resistance marker in place of the desired knockout gene or disruption of the target gene by incorporation of the resistance marker within the target gene. Alternatively, gene knock out can be achieved by incorporation of one or more stop codons, which lead to premature abortion of gene expression, or by insertions or deletions of single or multiple base pairs, which cause frameshift mutations and/or premature stop codons and/or deletions of functional protein domains rendering the protein dysfunctional, or a combination of two or more of these methods. In order to achieve complete functional gene inactivation, typically both genomic copies of a target gene are functionally inactivated.

Knock-out of the IRF3 and/or IRF7 genes can be performed by genetical engineering using methods established in the art, such as homologous recombination techniques, or techniques employing site- specific nucleases, including zinc-finger nucleases, transcription activator-like effector nuclease (TALENs) and clustered regularly interspaced short palindromic repeats (CRISPR) (Gaj, Thomas; Gersbach, Charles A.; Barbas, Carlos F., Trends in Biotechnology. 31 (7), p. 397-405).

Preferably, gene knock out is performed using the CRISPR/Cas9 system, which is a method for genome editing that uses target gene sequence-specific guide RNA complexed with a Cas9 protein (CRISPR/Cas9 system; M. R. O'Connell et al., Nature. Vol. 516 (7530), Dec. 2014, p. 263-266; C. Brandl et al., FEBS Open Bio, 5 (2015) 26-35). The Cas9 protein and guide RNA can be delivered to the cell via multiple transfection strategies such as plasmid-based, mRNA/gRNA based and protein/gRNA based.

By using, e.g., CRISPR/Cas9 mediated gene targeting, it becomes possible to functionally disrupt either single IRF3 and IRF7 genes, or both of them simultaneously, as multiple target-gene specific Cas9/gRNA can be applied to the cell at the same time. Preferably, the CRISPR/Cas9 system is used to functionally disrupt both IRF3 and IRF7 encoding genes simultaneously. The resulting double gene knock out prevents transcriptional activation of type I interferon genes. The absence of type I interferon prevents upregulation of antiviral genes in both the infected cell and the neighboring cells. The resulting absence of the antiviral state of the cells is used in the present invention to increase viral yields in cell culture.

Thus, the present invention provides a method of CRISPR-Cas9 mediated gene editing comprising the steps of:
(a) providing an MDBK cell and
(b) performing CRISPR-Cas9 mediated functional gene inactivation of the IRF3 and/or IRF7 encoding genes of the MDBK cell.

In an embodiment, the present invention is directed to a MDBK cell or to a method for producing a MDBK cell, in which both IRF3 and IRF7 genes are functionally inactivated by using the CRISPR/Cas9 gene targeting method.

In a further embodiment, the invention provides a cell culture comprising the MDBK cell as described herein. As used herein, the term “cell culture” encompasses the maintenance and/or propagation of cells and could be in vitro or ex vivo. As used herein, the phrase “ex vivo” refers to cells taken from an organism and subsequently maintained or propagated outside of that organism.

Methods of growing MDBK cells in cell culture are known in the art, and are, for example, available from ATCC. MDBK cells are typically grown in Eagle's Minimum Essential Medium (MEM) or Dulbecco's Modified Eagle Medium (DMEM). To make the complete growth medium, horse serum may be added to a final concentration of 10%. MDBK cells are conventionally passaged whenever they become confluent (see, e.g., L. Zhu et al., Veterinary Microbiology, 144 (2010) 51-57).

MDBK cells can be cultured in adherent conditions in, for example, tissue culture flasks, roller bottles or other tissue culture treated substrates. Procedures for such adherent cell cultures are well described in the art. For example, culture methods are described by the ATCC or the Cellbank Australia, which describe that the base medium is Eagle's Minimum Essential Medium, or a modification thereof. To make a complete growth medium, horse serum or bovine serum can be added to a final concentration of 10%.

Furthermore, it is possible to adapt such cultures in Animal Component Free conditions (ACF), where serum is replaced for a protein source that is not derived from animals. For MDBK, this is described by: T. Johnson, Sigma-Aldrich Corporation, St. Louis, Mo., USA: Serum-Free Systems for MDBK and MDCK Epithelial Cells.

Subculturing of adherent cell cultures can be achieved for example by using a fresh 0.25% trypsin, 0.03% EDTA solution, subsequent incubation, detachment of cells, addition of fresh medium to neutralize trypsin and dispension into new flasks. Alternatively, animal component free trypsin dissociation solutions consisting of recombinant trypsin or other peptidases can be used in combination with animal component free media. Cryopreservation can be performed by freezing MDBK cells in culture medium supplemented with 5-10% DMSO.

Typical culture conditions of MDBK are at 37° C. but may be increased to 38.6° C., the normal body temperature of a cow, and in an adequate humidified CO₂ environment in case carbonate-buffered culture media are used.

MDBK cells can be cultured in (e.g. animal serum free) dynamic conditions in roller bottles or on microcarrier beads in a suspension bioreactor.

Furthermore, MDBK cells can be grown in non-adherent conditions in shaker flasks in suspension and in suspension bioreactors.

Methods that allow large-scale production of cells enable optimization of live virus vaccine production. The suitability of MDBK cells as a substrate for virus production, e.g. BRSV growth, is, for example, described by F. R. Spilki et al., Journal of Virological Methods, 131 (2006) 130-133.

The cell culture of the present invention may be applicable to the production of all types of viruses, including DNA, RNA and DNA-RNA viruses, single-stranded (positive and negative sense) and double-stranded viruses, those with a circularized or linear genome, enveloped and non-enveloped viruses. In a preferred embodiment of the present invention, the virus is an enveloped virus. Non- limiting examples of viruses that can be grown on MDBK cells include, without being limited to, bovine parainfluenza virus 3 (bovine PIV), influenza A virus, porcine influenza virus (PIV), swine influenza virus (SIV), avian influenza virus, infectious bronchitis rhinotracheitisvirus (IBR), bovine herpesvirus 1 (BHV1), bovine viral diarrhea virus (BVDV), bovine coronavirus (BCV), bovine respiratory syncytial virus (BRSV), mumps virus, measles virus, members of the Bunyaviridae virus family and Schmallenberg virus.

In a further preferred embodiment, the virus is a bovine virus, including but without being limited to, BRSV, bovine diarrhea virus, bovine rhinotracheitis virus, bovine parvovirus, bovine adenovirus, bovine corona virus and bovine parainfluenza virus. In a particularly preferred embodiment, the virus is BRSV.

Therefore, in another embodiment the present invention provides a cell or cell culture as described herein for virus production. The term “virus production” in the context of the present invention typically means that the virus is replicated by using the cell or cell culture.

Thus, in a particular embodiment, the present invention is directed to an MDBK cell, in which both IRF3 and IRF7 genes are knocked out and wherein the cell is used for the production of BRSV.

In another embodiment, the present invention provides a method for the production of a virus, in particular for the production of BRSV, the method comprising the steps of:
(a) culturing the cell of the present invention,
(b) infecting the cell with a virus,
(c) allowing the virus to replicate, and
(d) optionally isolating the virus from the cell culture.

The resulting cell density of the cells in step (a) is not particularly limited, and depends on the culturing conditions (adherent, suspension) and culture medium. It is, for example, 1 to 100×10⁵ cells/mL, such as 2 to 90×10⁵ cells/mL, 3 to 80×10⁵ cells/mL, or 4 to 70×10⁵ cells/mL.

In step (b), the cell is infected with the virus. The concentration of the virus is not particularly limited, but it is, for example, 50 to 10,000 TCID 50/mL, 80 to 8,000 TCID 50/mL, or 90 to 2,000 TCID 50/mL. Multiplicity of infection (MOI) may be 0.00001 to 0.1, 0.0001 to 0.01, or 0.001 to 0.05.

In step (c), virus is allowed to replicate. In order to achieve replication, the cells infected with the virus are typically incubated under suitable culturing conditions. Conditions for the incubation are not particularly limited and may depend on the virus to be produced. For example, the culture temperature may be 32 ° C. to 40 ° C., 33 to 39 ° C., or 34-38. The culture period may be 12 hours to 6 days, 18 hours to 5 days, or 24 hours to 4 days. The carbon dioxide concentration may be 4 to 6%, such as about 5%. The oxygen concentration may be 2 to 10 ppm, for example 3 ppm.

In step (d), the virus may optionally be isolated, i.e. harvested, typically by separating the culture supernatant from the cells or by harvesting the entire culture.

Highly surprisingly, with the MDBK cell of the present invention, in which the IRF3 and/or IRF7 encoding genes are knocked out, it is possible to achieve higher virus titers compared to the wild type cell, i.e. a cell which does not contain the IRF3 and/or IRF7 gene knock out, but is otherwise completely impaired regarding the corresponding interferon response. Typical virus titers (nog TCID50/mL) which can be achieved in the present invention may be 5 to 10, preferably 6 to 9, further preferably 7 to 8. Surprisingly, it was found that with the MDBK cells of the present invention, in which the IRF3 and IRF7 genes are functionally inactivated, it is possible to achieve higher virus titers compared to a spontaneous type I IFN production-deficient MDBK cell line. Moreover, a genetically identical single cell clone, in which the IRF3 and IRF7 are functionally inactivated, did not show such increase; titers were comparable to a spontaneous type I IFN production-deficient MDBK cell line.

In another embodiment, the present invention provides a vaccine composition comprising the cell culture as described herein or a virus isolated from the cell culture and a pharmaceutically acceptable carrier.

A “vaccine” is well known to be a composition that has a medical effect, namely a prophylactic effect against a post-vaccination infection and/or against the signs resulting from such an infection. A vaccine comprises an immunologically active component, and a pharmaceutically acceptable carrier. The ‘immunologically active component’, in the present case comprises the virus produced by the cell of the present invention or a component derived therefrom such as a killed virus or a (recombinantly produced) subunit, or toxin thereof, etc. The vaccine is recognized by the immune system of the target human or animal, which induces a protective immunological response. The response may originate from the targets' innate- and/or from the acquired immune system and may be of the cellular- and/or of the humoral type.

A vaccine generally is efficacious in reducing the severity of an infection, for example by reducing the number of the pathogens, or shortening the duration of the pathogen's replication in a host animal. Also, or possibly as a result thereof, a vaccine generally is effective in reducing or ameliorating the (clinical) symptoms of disease that may be caused by such infection or replication, or by the target's response to that infection or replication.

In a preferred embodiment of the present invention, the vaccine is a vaccine against BRSV infection.

In another embodiment, the present invention provides a method for the preparation of a vaccine composition comprising a virus, the method comprising:
(a) culturing the cell of the present invention,
(b) infecting the cell with a virus,
(c) allowing the virus to replicate,
(d) optionally isolating the virus from the cell culture, and
(e) mixing the virus or the cell culture infected with the virus with a pharmaceutically acceptable carrier.

The pharmaceutically acceptable carrier is not particularly restricted and can be any carrier suitable for use in virus vaccines. Examples of pharmaceutically acceptable carriers that are suitable for use in a vaccine according to the invention are, for example, sterile water, saline, and aqueous buffers such as PBS. In addition, a vaccine according to the invention may comprise other additives such as adjuvants, stabilizers, antioxidants and others.

In a preferred embodiment, the pharmaceutically acceptable carrier comprises a natural deep-eutectic solvent (NADES) having a water activity of less than about 0.8. For a description of NADES, reference is made to European patent application EP 17 210 395, which is hereby incorporated by reference in its entirety.

Use of NADES as pharmaceutically acceptable carrier has been found to be able to stabilize enveloped viruses in liquid vaccine compositions. Enveloped viruses include virus families with RNA genome of: Corona-, Flavi-, Toga-, Arena-, Bunya-, Filo-, Orthomyxo-, Paramyxo-, Pneumo-, Rhabdo-, Reo- and Retroviruses. In a preferred embodiment, the enveloped virus is a Pneumovirus and most preferably is BRSV.

NADES is particularly useful in liquid vaccine compositions. It has been found that NADES stabilize liquid virus vaccines, in particular live virus vaccines. It is thus possible to provide the vaccines of the present invention with improved stability, thereby eliminating the need for freezing or refrigeration. Instead, the liquid vaccine composition including the NADES carrier is sufficiently stable in liquid form with sensitive viruses, and upon prolonged storage.

An advantageous characteristic of the liquid vaccine composition comprising a NADES, is that the water that is present in the vaccine is tightly bound in the structure of the NADES. The result of this is that the amount of water that is available for chemical or biological processes that could influence the stability of the virus, is very limited. This feature is commonly expressed in the value of the water activity, indicated by the symbol: a_{w}. The water activity varies between an upper limit of 1.0 for pure water, and the lower limit of 0. Water activity is commonly measured by comparing (at the same temperature) the vapour pressure of a test composition, relative to that of pure water and to a number of saturated salt-solutions of known water activity. This is described in different handbooks, reviews and manuals, such as for example on the conservation of fruits and vegetables in the FAO agricultural service bulletin no. 149 (Cánovas et al, FAO, Rome, 2003, ISBN 92-5-104861-4); and a review is in ‘Fundamentals of water activity’, Decagon Devices Inc., Washington, 2015 (http://pdf.directindustry.com/pdf/decagon-devices-inc/fundamentals-water-activity/64142-634433.html). Equipment and procedures to measure water activity are well known and available, for instance by using headspace pressure analysis.

At a water activity less than 0.8 growth of most bacteria is stopped; at less than 0.7 growth of most yeasts and moulds is stopped, and at a water activity less than 0.4 most enzyme activity is effectively stopped. It was found that effective liquid vaccine compositions according to the invention can be prepared with water activities between 0.8 and 0.1. Therefore, the water activity of the liquid vaccine composition is preferably less than 0.7; more preferably less than 0.6, 0.5, 0.4, 0.3, or even less than 0.2, in this order of preference.

For the invention, the indicated water activity refers to the water activity of the liquid vaccine composition according to the invention in the form of a final product, for example such as is offered by a commercial producer, and in which form it can be stored for prolonged time.

Therefore, the invention further provides a liquid vaccine composition, wherein one, or more, or all of the conditions apply, selected from the group consisting of:
the vaccine has a water activity of less than about 0.8, and comprises a NADES;
the water activity of the vaccine is less than about 0.7; more preferably less than about 0.6, about 0.5, about 0.4, or even less than about 0.3, in this order of preference;
the NADES consists of an organic salt and a polyol;
in the NADES the organic salt is selected from salts of: betaine, proline, carnitine, and choline;
the carnitine is L-carnitine;
the choline is choline-chloride;
the polyol is a sugar or a sugar-alcohol;
the sugar is selected from: fructose, maltose, sucrose, glucose and trehalose;
the sugar-alcohol is selected from: glycerol, xylitol, mannitol, and sorbitol; more preferred the sugar-alcohol is selected from the group of: glycerol, xylitol, and sorbitol;
the sorbitol is D-sorbitol;
the polyol is selected from: sucrose, glycerol, xylitol, and sorbitol;
the organic salt is selected from salts of: betaine, proline, carnitine, and choline; and the polyol is selected from fructose, maltose, sucrose, trehalose, glycerol, xylitol, mannitol, and sorbitol;
preferably the organic salt is selected from: betaine, proline, and choline; and the polyol is selected from sucrose, glycerol, xylitol, and sorbitol; more preferably the organic salt is selected from proline and choline; and the polyol is selected from glycerol, xylitol, and sorbitol;
in the liquid vaccine composition according to the invention, the molar ratio between the organic salt and the polyol, as defined herein, is between 1:4 and 4:1; preferably the molar ratio between the organic salt and the polyol, as defined herein, is between 1:3 and 3:1, or even is between 1:2 and 2:1;
the water content in the liquid vaccine composition according to the invention is less than about 40% w/v, less than about 30, 25, 20, 15, 10, 8, 7, 6, or even 5% w/v, in that order of preference;
the water content in the vaccine is between 50 and 0.5% w/v; between 40 and 1% w/v; between 30 and 1.5% w/v; between 20 and 2% w/v; or even between 10 and 3% w/v, in this order of preference; and
the liquid vaccine composition according to the invention may comprise a preservative, such as thimerosal, merthiolate, phenolic compounds, and/or gentamicin. Preferably no preservative is employed.

A vaccine according to the invention may comprise the virus according to the invention in live, attenuated live or inactivated form. Preferably, the virus according to the invention is in live or live attenuated form. Therefore, the method for the preparation of a vaccine according to the present invention may optionally further contain a step of attenuating or inactivating the virus as produced according to the present invention.

Attenuated live virus vaccines, i.e. vaccines comprising the virus according to the invention in a live attenuated form, have the advantage over inactivated vaccines that they best mimic the natural way of infection. In addition, their replicating abilities allow vaccination with low amounts of viruses; their number will automatically increase until it reaches the trigger level of the immune system. From that moment on, the immune system will be triggered and will finally eliminate the viruses. A live attenuated virus is a virus that has a decreased level of virulence when compared to virus isolated from the field. A virus having a decreased level of virulence is considered a virus that does not cause the typical symptoms of viral infection. Therefore, one preferred form of this embodiment of the invention relates to a vaccine comprising a virus according to the invention wherein said virus is in a live attenuated form.

Attenuated viruses can e.g. be obtained by growing the viruses according to the invention in the presence of a mutagenic agent, followed by selection of virus that shows a decrease in progeny level and/or in replication speed. Many such agents are known in the art.

Inactivated vaccines are, in contrast to their live attenuated counterparts, inherently safe, because there is no rest virulence left. In spite of the fact that they usually comprise a somewhat higher dose of viruses compared to live attenuated vaccines, they may e.g. be the preferred form of vaccine in subjects that are suffering already from other diseases.

Therefore, another embodiment relates to a vaccine comprising a virus according to the invention wherein said virus is in an inactivated form. In a preferred embodiment, the virus is BRSV.

The standard way of virus inactivation is a classical treatment with formaldehyde. Other methods well-known in the art for inactivation are UV-radiation, gamma-radiation, treatment with binary ethylene-imine, and thimerosal.

### EXAMPLES

The present invention will be described with reference to the following non-limiting examples and figures:

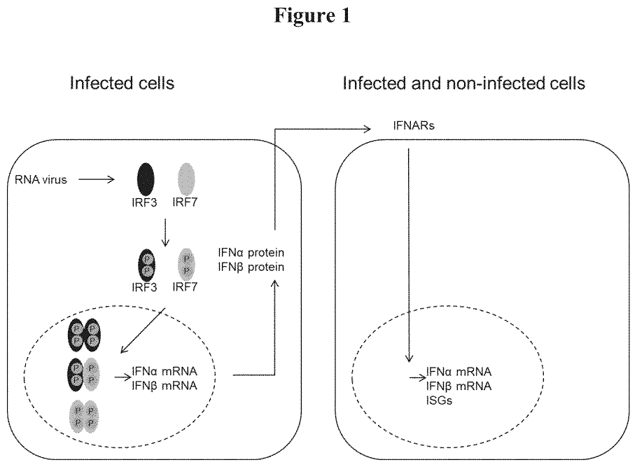

FIG. 1: Schematic representation of the Interferon-regulatory factors in positive-feedback regulation of type I interferon genes (illustration similar to: Honda and Taniguchi, Nature Reviews Immunology 6, 644-658, 2006).

Honda and Taniguchi describe the stimulation of IRF3 and IRF7 in virus-infected cells, and the subsequent autocrine and paracrine feedback loops. In the early phase of infection with an RNA virus, interferon (IFN)-regulatory factor 3 (IRF3) and IRF7 are phosphorylated on specific serine residues, resulting in the homodimerization or heterodimerization of IRF3 and IRF7. These dimers then translocate to the nucleus and induce the expression of chemokines (not shown), as well as small amounts of IFNβ and IFNα (figure left). Secreted IFNs then bind and activate the type I IFN receptor (a heterodimer of IFNAR1 and IFNAR2) in an autocrine or paracrine manner. This interaction leads to the expression of large amounts of IFNβ and many of the IFNα proteins. Therefore, a positive- feedback loop (that is, type-I IFN-dependent type I IFN gene induction) is operational. Furthermore, a large amount of Interferon Stimulated genes (ISGs) is induced, which induce an antiviral state in the cell. An example of such an ISG is MX1.

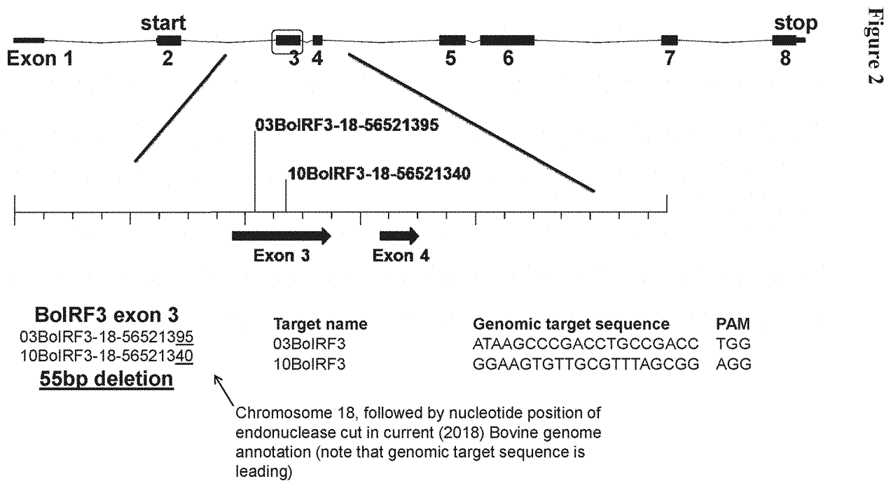

FIG. 2: CRISPR/Cas9 gRNA design for functional inactivation of IRF3

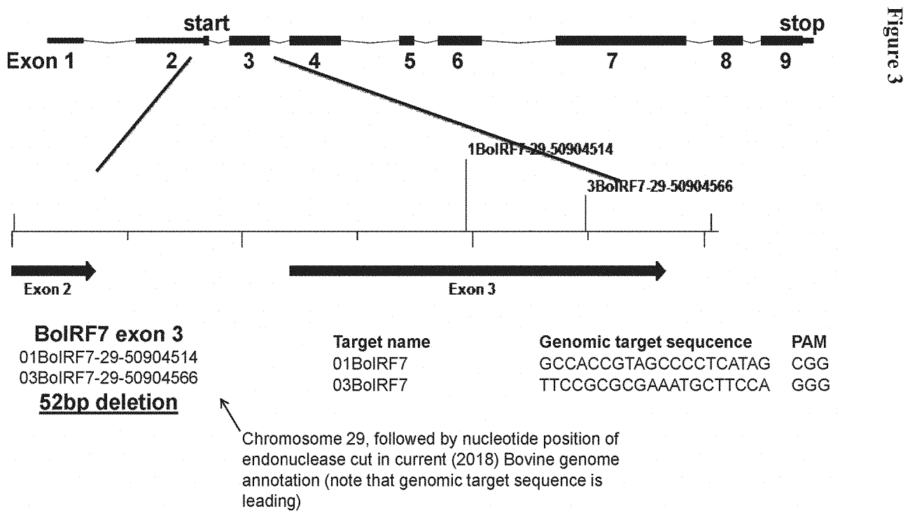

FIG. 3: CRISPR/Cas9 gRNA design for functional inactivation of IRF7

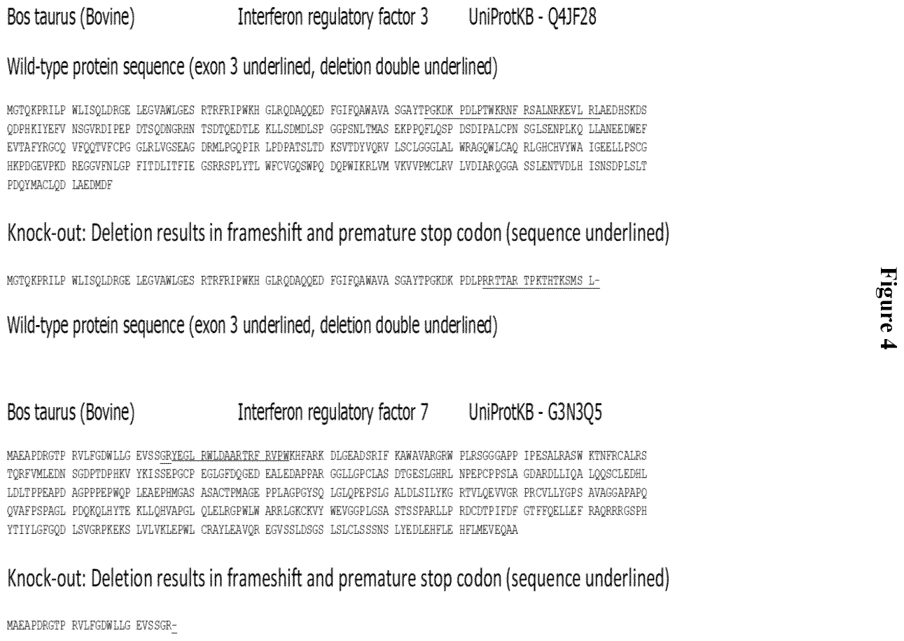

FIG. 4: MDBK #2 single cell clone sequence analysis: confirmation of IRF3/IRF7 gene knock-out

### Example 1

Preparation of MDBK ΔIRF3/IRF7 double knock out mutant cells (designated as MDBK cell clones #1 and #2) using CRISPR/Cas9

MDBK cell clones #1 and #2 are clonal cell lines derived from MDBK 42/E9 (CCT103). In each of the clones, both genomic copies of the IRF3 gene and both genomic copies of the IRF7 gene have been functionally inactivated using CRISPR/Cas9 mediated genome editing.

Parental cell line MDBK 42/E9 was cultured in animal component free (ACF) culture medium (T. Johnson, Sigma-Aldrich Corporation, St. Louis, Mo., USA: Serum-Free Systems for MDBK and MDCK Epithelial Cells.)+0.1% Poloxamer 188 and passaged using cell dissociation reagent TrypLE (Animal component-free, recombinant trypsin that replaces porcine trypsin) in PBS+0.01% EDTA. Cells were cultured in adherent conditions in Corning Biocoat collagen I culture flasks. Cells were cultured at 38° C. and 5% CO₂ in a humidified incubator.

The Cas9/RNA complexes were prepared as described in the Alt-R CRISPR user guide (“Alt-R CRISPR-Cas9 System:Cationic lipid delivery of CRISPR ribonucleoprotein complexes into mammalian cells”, 2018, Integrated DNA Technologies, Inc.). The Cas9/gRNA complexes were delivered to the cell by electroporation after preparation in the test tube according to the Alt-R CRISPR user guide. The genomic target sequences are listed in Table 1.

TABLE 1
CRISPR-Cas9 gRNA design

| Target name | Genomic target sequcence | PAM |
|---|---|---|
| 03BoIRF3 | ATAAGCCCGACCTGCCGACC | TGG |
| 10BoIRF3 | GGAAGTGTTGCGTTTAGCGG | AGG |
| 01BoIRF7 | GCCACCGTAGCCCCTCATAG | CGG |
| 03BoIRF7 | TTCCGCGCGAAATGCTTCCA | GGG |

Transfection was carried out via electroporation using the Lonza Nucleofector 4D equipment in buffers and cuvettes as supplied by the manufacturer. The electropulse was initially optimized to achieve maximal transfection efficiency in MDBK cells, which was monitored by transfection of a Alexa488-labeled Cas9 protein. After electroporation of 1×10⁶ cells, these were allowed to recover and subsequently cultured in 6-well plates (collagen-I coated). The cell pool was transfected with total of 4 sgRNA-Cas9 complexes; 2 targeting IRF3 and 2 targeting IRF7. After reaching confluency, the cell pool was passaged to 2×T25 flask (Collagen I coated). At confluency, these cells were cryopreserved in culture medium supplemented with FBS and DMSO.

One ampoule of the cryopreserved cells was started up in culture medium as described above to initiate single cell cloning. The cells were counted and diluted to a density of 1 cell per 100 μL, and subsequently 100 μL was dispensed per well of a 96-well plate (Collagen I coated). After attachment, visual inspection was carried out to check if single cell cultures indeed had been established. Single cells were allowed to grow and single-cell derived cultures were passaged to 48-well plates, subsequently to 6-well plates, T25s and larger culture substrates to allow further expansion of the cultures. Further expanded and cryopreserved as described above.

The single cell clones #1 and #2 cells were genotyped by PCR to check if the designed deletions were present. The designed deletions were a 55 bp deletion in the IRF3 gene (exon 3) on chromosome 18, and a 52 bp deletion in the IRF7 gene (exon 3) on chromosome 29 (see FIGS. 2 and 3). Analysis of gene knock-out by gene sequencing confirmed the introduction of premature stop codons (FIG. 4).

### Example 2

Testing of functionality of the Interferon type 1 signaling pathway in MDBK CCT094, MDBK 42/E9, MDBK #1 and MDBK #2 cell lines

TABLE 2
Overview of MDBK cell lines used in the experiments:

| | |
|---|---|
| MDBK CCT094 | Current BRSV production cell line |
| (Marburg) | |
| MDBK 42/E9 | MDBK 42 obtained by Intervet International |
| | BV from the University of |
| | Hannover in 1990. MDBK 42/E9 is a |
| | single cell clone selected based on optimal |
| | growth of Bovine coronavirus |
| MDBK #1 | MDBK 42/E9 with AIRF3/IRF7 |
| | as described in this application |
| MDBK #2 | MDBK 42/E9 with AIRF3/IRF7 |
| | as described in this application |

Experiment 1

This experiment was set up to measure relative gene expression levels of ISGs (Interferon-stimulated genes) 24 hrs after Poly (I:C) transfection. This experimentally addresses functionality of the first phase of the innate immune response after pathogen recognition by TLRs in virus infected cells (see FIG. 1). Poly (I:C) is synthetic dsRNA that mimics viral RNA infection.

Cell lines tested:
MDBK CCT094
MDBK 42/E9

Reporter genes:
Bovine RNA polymerase 2 (reference gene for normalization)
Bovine IRF7 Interferon regulatory factor 7 (Interferon-stimulated gene, ISG)
BoMX1 Interferon-induced GTP-binding protein Mx1: a classical ISG that is induced upon BRSV infection (see Experiment 3)

Rationale:

Type I Interferon production by infected cells and subsequent binding to IFNARs (autocrine/paracrine) leads to the activation of IFN-stimulated gene factor 3 (ISGF3; a heterotrimer of signal transducer and activator of transcription 1 (STAT1), STAT2 and IRF9), which translocates to the nucleus and induces the transcription of the IRF7 gene. Activation of newly synthesized IRF7 leads to the expression of large amounts of IFNβ and many of the IFN α proteins. Therefore, IRF7 is part of a positive-feedback loop (that is, type-I-IFN-dependent type I IFN gene induction) and thus a good mRNA for readout of the type I Interferon response (Honda and Taniguchi, Nature Reviews Immunology 6, 644-658, 2006)

Method:

Cells were cultured (adherent) in ACF medium on collagen coated surfaces. At t=−24 hrs, cells were passaged and seeded for the experiment so that the monolayer reached a density of about 70% confluency at the time of Poly (I:C) transfection. At t=0 hrs, cells were transfected with Poly (I:C) (100 ng, 500 ng synthetic dsRNA per 6-well transfection using FUGENE 6). A non-transfected control was taken along as a reference. At 24 hrs, medium was removed from the cells, and cells were harvested in RLT buffer (QIAGEN) for subsequent RNA analysis.

qPCR analyses were set up for the RNA polymerase 2, IRF7 and MX1 (primer sets in Table 3 below; IDT: integrated DNA technologies). RNA was extracted from the samples using the Magnapure® extraction method (kit name: DNA/Viral NA Small volume v2.0 Protocol name: Viral NA Plasma external lysis small volume 3.1; Roche). Relative expression was calculated using the Normalized gene expression in MDBK cell lines 24 hours after Poly (I:C) transfection expression, calculated as the 2\hat{ }ΔΔCt fold change, as described by: Pfaffl, Nucleic Acids Research, Vol. 29, No. 9 00; 2001.

TABLE 3
Primers used for qPCR analysis

| Amplicon | Name Oligo | Sequence 5′-3′ | Label (5′) |
|---|---|---|---|
| Bos Taurus | BoIRF3 wt reg FW | CAAGGTCTAAGTCATCTTCCCC | — |
| IRF3 | BoIRF3 wt reg REV | GGACACTCGAAATCCCATCC | — |
| | 6FAM-BoIRF3-In2-fw | CAAGGTCTAAGTCATCTTCCCC | 6FAM |
| Bos Taurus | BoIRF7 wt reg FW | CTTTGCGGAGGGACCAATG | — |
| IRF7 | BoIRF7 wt reg REV | GGGCAGAGCTAGAGTCTCAC | — |
| | 6FAM-BoIRF7-ex2- | CTTTGCGGAGGGACCAATG | 6FAM |
| | FW | | |
| BoPOLR2A | PrimeTime Assay | AGAGCAATCGTCCAGTCATG | — |
| | (IDT) | GTGGACAGGAACATCAGGAG | — |
| | | CGCTCCAGG/ZEN*/AAGACATC | 6FAM/IABkFQ |
| | | CCTCTTG | |
| BoMx1 | PrimeTime Assay | TGACCCTGATAGACCTTCCTG | — |
| | (IDT) | GTTGATGGTCTCCTGCCTAAG | — |
| | | CAACCAGCC/ZEN*/ACCCGACA | 6FAM/IABkFQ |
| | | TTGAA | |

*ZEN is a quencher in the probe used for the qPCR (available rom Integrated DNA Technologies Inc., Coralville, Iowa, USA)

TABLE 4
Results of normalized IRF7 transcription in MDBK
cell lines 24 hours after Poly (I:C) transfection

| | Amount of Poly (I:C) | IRF7 transcript level |
|---|---|---|
| Cell clone | transfection [ng] | fold-change (2\hat{ }ΔΔCt) |
| MDBK CCT094 | 100 | 0.9 |
| | 500 | 1.3 |
| MDBK 42/E9 | 100 | 8.3 |
| | 500 | 12.9 |

It could be shown that compared to the non-treated control cells, transcription levels of BoIRF7 were upregulated 24 hours after Poly (I:C) transfection in MDBK 42/E9, but not in MDBK CCT094 (Table 4).

TABLE 5
Results of normalized Mx1 transcription in MDBK
cell lines 24 hours after Poly (I:C) transfection

| | Amount of Poly (I:C) | IRF7 transcript level |
|---|---|---|
| Cell clone | transfection [ng] | fold-change (2\hat{ }ΔΔCt) |
| MDBK CCT094 | 100 | 1.2 |
| | 500 | 16 |
| MDBK 42/E9 | 100 | 206 |
| | 500 | 189 |

It could be shown that compared to the non-treated control cells, transcription levels of BoMX1 were upregulated 24 hours after Poly (I:C) transfection in MDBK 42/E9 about 200-fold. In MDBK CCT094 the upregulation was only 16-fold, only when induced with 500 ng Poly (I:C) per well. With 100 ng Poly (I:C) was upregulated 1.2-fold (Table 5).

Conclusions:
Poly (I:C) transfection in Bovine MDBK 42/E9 cells results in activation of the type I Interferon signaling pathway, or innate immunity pathway.
MDBK 42/E9 cells respond to both doses of Poly (I:C).
MDBK CCT094 does not show induction of the IRF7 ISG after transfection with Poly (I:C) (both doses), and only a limited increase of expression of MX1 at a high Poly (I:C) transfection dose. The transcript level increase of MX1 is about 10-fold lower than in MDBK 42/E9, which indicates that MDBK CCT094 is partially defective in inducing a type I interferon response upon dsRNA recognition.

Experiment 2

Test of the Type I Interferon positive feedback loop.

It was subsequently investigated whether active Type I interferon proteins were present in the culture supernatant of cultures of MDBK 42/E9 and MDBK CCT094. This experiment addresses functionality of the second phase of the innate immune response after pathogen recognition by TLRs in virus infected cells (see FIG. 1; autocrine and paracrine signaling via Interferons). For this purpose, supernatants from MDBK CCT 094 and MDBK 42E9 cells were harvested at 24 hours after Poly (I:C) transfection and subsequently transferred to freshly cultured cells.

Protocol
T=−24 hrs Seed cells (2 cell lines: MDBK 42/E9 and MDBK CCT094)
T=0 hrs No treatment/Transfection of 500 ng Poly (I:C)

T=24 hrs Harvest supernatant. Add this supernatant (no treatment/Poly (I: C)) to fresh monolayers of both MDBK 42/E9 and MDBK CCT094 cells.

T=6 hours after supernatant transfer: Harvest cells and measure MX1 ISG mRNA levels by qPCR

MDBK 42/E9 cells at T=24 hrs:

Supernatant “no treatment” was added to MDBK-MB CCT094 and MDBK 42/E9

Supernatant “500 ng Poly (I:C)” was added to: MDBK-MB CCT094 and MDBK 42/E9

MDBK-MB CCT094 cells at T=24 hrs:

Supernatant “no treatment” was added to: MDBK-MB CCT094 and MDBK 42/E9

Supernatant “500 ng Poly (I:C)” was added to: MDBK-MB CCT094 and MDBK 42/E9

T=30 hrs (24+6) Lyse cells in RLT-buffer (6 hrs after supernatant transfer)

RNA was extracted from the samples using the Magnapure extraction method as described above. Relative expression was calculated using the Normalized gene expression in MDBK cell lines 24 hours after PolyI:C transfection expression fold change (2\hat{ }ΔΔCt) as described above.

Results:

Transferred supernatant from non-transfected cultures to fresh MDBK 42E9 or MDBK CCT094 cells did not induce upregulation of BoMX1 (1.0).

Transferred supernatant from Poly (I:C) transfected MDBK CCT094 cultures to fresh MDBK CCT094 cells did not result in upregulation of BoMX1 (1.0).

Transferred supernatant from Poly (I:C) transfected MDBK 42/E9 cultures to fresh MDBK CCT094 cells resulted in a 163-fold upregulation of BoMX1.

Transferred supernatant from Poly (I:C) transfected MDBK 42/E9 cultures to fresh MDBK 42/E9 cells resulted in a 208-fold upregulation of BoMX1.

Transferred supernatant from Poly (I:C) transfected MDBK CCT094 cultures to fresh MDBK 42/E9 cells resulted in a marginal 3.9-fold upregulation of BoMX1.

TABLE 6
Results of normalized Mx1 transcription in MDBK
cell lines 6 hours after addition of culture
supernatant from cells transfected with Poly (I:C)
(500 ng-24 hrs), Transcript level fold-change (2\hat{ }ΔΔCt)

| | Supernatant of Cell | MX1 transcript level |
|---|---|---|
| Test Cell line | clone | fold-change (2\hat{ }ΔΔCt) |
| MDBK CCT094 | MDBK CCT094 | 1.0 |
| | MDBK 42/E9 | 163 |
| MDBK 42/E9 | MDBK CCT094 | 3.9 |
| | MDBK 42/E9 | 208 |

Conclusion:

This experiment convincingly shows that the Interferon-binding and signaling part of the innate immune system (FIG. 1, right part, positive feedback loop) is present and functional in both MDBK CCT094 and MDBK 42/E9, and again shows that MDBK CCT094 is defective in the first part (FIG. 1, left part) of the innate immunity signaling pathway, the initial induction of a Type I interferon response upon pathogen recognition.

Based on these experiments it is now evident that MDBK-MB CCT094 cells have a defective innate immunity signaling pathway with no interferon induction upon pathogen recognition but possess a functional type 1 interferon positive feedback loop.

Experiment 3

No induction of BoMX1 in ΔIRF3/IRF7 cell lines MDBK #1 and MDBK #2 after BRSV infection.

Successful inactivation of the Type I Interferon signaling system was confirmed by determining the MX1 ISG induction upon infection of cells with the BRSV_{Jencine} strain at different MOIs.

Subconfluent monolayers of MDBK 42/E9, MDBK #1 and MDBK #2 were infected with BRSV_{Jencine} strain at MOIs 0.01, 0.01 and 0.05. Gene expression fold-change was measured as described above at 1, 2, 3 and 4 days after virus infection. The results in Table 7 show that MX1 gene induction upon BRSV infection is greatly impaired due to the functional inactivation of the IRF3 and IRF7 genes.

TABLE 7
Results of normalized Mx1 transcript levels in MDBK 42/E9,
MDBK #1 and MDBK #2 cell sat different
time points after infection with BRSV_{Jencine} strain (3 MOIs).
Data are expressed as transcript level fold-change (2\hat{ }ΔΔCt).

| | Days post | MXI transcript level | | |
|---|---|---|---|---|
| Test | infection | fold-change (2\hat{ }ΔΔCt) | | |
| Cell line | (dpi) | M.O.I: 0.01 | M.O.I: 0.05 | M.O.I: 0.001 |
| MDBK 42/E9 | 1 | 1.5 | 1.0 | 1.7 |
| | 2 | 40.5 | 30.9 | 24.3 |
| | 3 | 38.9 | 59.7 | 34.8 |
| | 4 | 5.1 | 10.3 | 9.6 |
| MDBK #1 | 1 | 0.9 | 1.1 | 0.7 |
| | 2 | 2.3 | 0.9 | 1.0 |
| | 3 | 1.5 | 1.4 | 1.6 |
| | 4 | 0.7 | 0.5 | 0.4 |
| MDBK #2 | 1 | 3.7 | 4.0 | 3.3 |
| | 2 | 9.6 | 0.9 | 1.0 |
| | 3 | 0.8 | 1.1 | 1.3 |
| | 4 | 0.3 | 0.4 | 0.6 |

Conclusion:

This experiment confirms that the Interferon-binding and signaling part of the innate immune system (FIG. 1, right part, positive feedback loop) is present and functional in MDBK 42/E9. In addition, this experiment convincingly shows that the initial induction of a Type I interferon response (first part of the innate immunity signaling pathway, FIG. 1, left part) upon pathogen recognition is not present in both ΔIRF3/IRF7 mutant cell lines MDBK #1 and MDBK #2.

### Example 3

Production of BRSV_{Jencine} in ΔIRF3/IRF7 mutant cell lines MDBK #1 and MDBK #2 and parental cell line MDBK 42/E9 in shaker flasks

Single cell clones of MDBK #1 and MDBK #2 and parental cell line MDBK 42/E9 were expanded in adherent culture conditions in roller bottles. Cells were cultured in animal component free (ACF) culture medium (T. Johnson, Sigma-Aldrich Corporation, St. Louis, Mo., USA: Serum-Free Systems for MDBK and MDCK Epithelial Cells.)+0.1% Poloxamer 188 and passaged using cell dissociation reagent TrypLE (Animal component-free, recombinant trypsin that replaces porcine trypsin, ThermoFisher) in PBS+0.01% EDTA. Roller bottles were coated with Intervet Coat I (Recombinant Human Collagen Type 1; 0.25 μg/cm²).

Subsequently, cells were passaged in ACF culture medium in shaker flasks. For this purpose, 250 ml shaker flasks with 125 ml culture medium were used, with cell densities between 0.5*10⁶ cells/mL to 1*10⁶ cells/mL. The culture was incubated at 37° C. and 5% CO₂ on a platform shaker at 150 rpm. The cell suspensions were passaged at least twice before use in BRSV_{Jencine} virus culture, at 4-day intervals. In the second experiment, also MDBK CCT094 was used.

For virus culture on MDBK CCT094, MBDK 42/E9, MDBK #1 and MDBK #2, BRSV_{Jencine} strain (MSD BRSV vaccine virus) was used. The virus batch used in the experiments was cultured on MDBK CCT094 in virus medium+5% Donor Horse Serum (DHS).

Prior to virus culture, cells were harvested from suspension cultures and resuspended in medium+5% DHS+0.1% poloxamer at cell densities of 0.5*10⁶ cells/mL and 1.0*10⁶ cells/mL in 250 mL shaker flasks with vented cap in a volume of 100 mL (experiment 1) or in 125 mL shaker flasks with vented cap in a volume of 50 mL (experiment 2). Virus was inoculated at a MOI of 0.01 TCID50/cell, and the culture was conducted at 36° C. in a humidified CO₂ incubator. Cultures were harvested at day 4.

Culture harvests were titrated on adherent MBDK cells. Titration is used to quantify the amount of infectious BRSV in bulk antigen samples. Serial tenfold dilutions of test samples were made in medium and were placed on microtiter plates pre-seeded with MDBK CCT094 cells. After an incubation period of 5-7 days at 37° C. in a humidified CO₂ atmosphere, the monolayers were examined for the presence or absence of BRSV. The microtiter plates were fixated, and subsequently an immune-peroxidase monolayer assay (IPMA) was performed. For this purpose, a peroxidase- labelled BRSV-specific monoclonal antibody in combination with a substrate was used for detection, which results in a colored precipitate that was scored visually. The viral titer was calculated in accordance with the method of Spearman and Kärber and was expressed in 10Log TCID50/mL. The positive control in the titration experiments was a BRSV harvest with a known titer. All samples were tested in duplicate, the positive control in triplicate.

Experiment 1

BRSV_{Jencine} virus batch 01G16, antigen after concentration, titer=7.4 Log10 TCID50/mL cultured on MDBK CCT094.

Cell lines tested:

MDBK 42/E9 (parental cell line)

MDBK #1 (ΔIRF3/IRF7)

MDBK #2 (ΔIRF3/IRF7)

Two cell densities: 0.5*10⁶ cells/mL and 1.0*10⁶ cells/mL, conducted in 250 mL shaker flasks (100 mL culture volume)

Results:

TABLE 8
BRSV titers at day 4 post infection in MDBK 42/E9,
MDBK #1 and MDBK #2

| | | Titer (¹⁰log | Increase vs. |
|---|---|---|---|
| Cell line | Cell density | TCID₅₀/ml) | MDBK 42/E9 |
| MDBK42/E9 | 0.5 * 10⁶ cells/mL | 6.60 | — |
| MDBK42/E9 | 1.0 * 10⁶ cells/mL | 6.65 | — |
| MDBK #1 | 0.5 * 10⁶ cells/mL | 7.20 | 0.6 |
| MDBK #1 | 1.0 * 10⁶ cells/mL | 7.25 | 0.6 |
| MDBK #2 | 0.5 * 10⁶ cells/mL | 7.65 | 1.0 |
| MDBK #2 | 1.0 * 10⁶ cells/mL | 7.85 | 1.2 |

The results show that functional deletion of IRF3 and IRF7 results in increased BRSV titers at day 4 post infection.

Experiment 2

BRSV_{Jencine} virus batch 01G16, antigen after concentration, titer=7.4 Log10 TCID50/mL cultured on MDBK CCT094, same batch as used in Experiment 1.

Cell lines tested:

MDBK 42/E9 (parental cell line)

MDBK CCT094 (MDBK with dysfunctional type I IFN signaling—see Example 2))

MDBK #1 (ΔIRF3/IRF7)

MDBK #2 (ΔIRF3/IRF7)

One cell density: 1.0*10⁶ cells/mL, conducted in 125 mL shaker flasks (50 mL culture volume)

TABLE 9
BRSV titers at day 4 post infection in MDBK 42/E9,
CCT094, MDBK #1 and MDBK #2

| | | Titer (¹⁰log | Increase vs. |
|---|---|---|---|
| Cell line | Cell density | TCID₅₀/ml) | MDBK 42/E9 |
| MDBK42/E9 | 1.0 * 10⁶ cells/mL | 5.6 | — |
| CCT094 | 1.0 * 10⁶ cells/mL | 5.8 | 0.2 |
| MDBK #1 | 1.0 * 10⁶ cells/mL | 6.7 | 1.1 |
| MDBK #2 | 1.0 * 10⁶ cells/mL | 7.3 | 1.7 |

Conclusions:

The results again show that functional deletion of IRF3 and IRF7 from MDBK 42/E9 results in increased BRSV titers at day 4 post infection. In this experiment, the current production cell line MDBK CCT094 was taken along, because this cell line also contains a dysfunctional Type I interferon signaling system. The expected effect of functional deletion of IRF3 and IRF7 from MDBK 42/E9 cells was a titer increase for BRSV to levels at maximum comparable to the MDBK CCT094.

Surprisingly and unexpectedly, however, the viral titers of the IRF3/IRF7 deficient mutant cell lines MDBK #1 and #2 show much higher titers than CCT094, a cell line that has spontaneously lost the ability to evoke a type I interferon response upon viral infection.

Therefore, the IRF3/IRF7 deficient mutant cell lines MDBK #1 and #2 can beneficially be used for virus production, in particular for growing viruses for vaccine production. Since these cell lines have a stably and permanently inactivated type I interferon response, these cell lines can beneficially be used for virus production achieving higher viral yields compared to a wild type parental cell line and even compared to a cell line, that has spontaneously lost the ability to evoke a type I interferon response upon viral infection.

### Example 4

Preparation of a MDBK IRF3 single knock out cell clone and a MDBK IRF7 single knock out cell clone was conducted as described in Example 1 by means of including only one of the respective sgRNA sets directed at the genetic target to be modified (i.e. only the IRF3 or IRF7 sgRNAs; Table 1). Subsequently, MDBK single cell clones (designated as cell clones MDBK cell clone #3 and MDBK cell clone #4) were established using the CRISPR/Cas9 platform as described in Example 1, and subsequent single cell cloning.

In cell clone #3, both genomic copies of the IRF3 gene have been functionally inactivated. In cell clone #4, both copies of the IRF7 gene have been functionally inactivated, which was confirmed by sequencing analysis of the targeted alleles. Both clones were subsequently tested for BRSV growth as described in Example 3.

The ΔIRF3 and ΔIRF7 cell lines and reference wild-type cell line MDBK 42/E9 (CCT103) were cultured in shaker flasks. Cells were passaged in animal component free (ACF) culture medium in shaker flasks as described in Example 3. The cell suspensions were passaged and are used for BRSV_{Jencine} strain virus culture.

The goal of the experiment was to compare yields of virus culture of BRSV_{Jencine} on the ΔIRF 3 and ΔIRF 7 cell lines to the reference CCT103 in shaker flasks at starting cell density 1.0*10⁶ cells/mL. Virus batch: BRSV_{Jencine} virus batch 01G16, antigen after concentration, titer=7.4 Log10 TCID₅₀/mL cultured on MDBK CCT094.

Cell lines tested:

MDBK 42/E9 (CCT103)

MDBK ΔIRF3 (MDBK #3)

MDBK ΔIRF7 (MDBK #4)

Method:

Cell density: 1.0*10⁶ cells/mL, conducted in 125 mL shaker flasks (50 mL culture volume) MOI: 0.01

Temp: 36° C. in CO₂ incubator

Sampling: V+3 (day 3 after infection), subsequent titrations as described in Example 3.

Results:

| | Titer (10log TCID₅₀/mL) | | |
|---|---|---|---|
| Day | CCT103 | ΔIRF3 | ΔIRF7 |
| V + 3 | 6.7 | 7.3 | 7.4 |

Also for the single knock out mutants the titers were higher in the ΔIRF3 and ΔIRF7 cell lines compared to CCT103.

1. A Madin-Darby bovine kidney (MDBK) cell, characterized in that the cell is genetically engineered by which the interferon regulatory factors (IRF) IRF3 and/or IRF7 encoding genes are functionally inactivated.

2. The MDBK cell of claim 1, IRF3 and IRF7 encoding genes are functionally inactivated.

3. The MDBK cell of claim 1, which is infected with a virus.

4. The MDBK cell of claim 3, wherein the virus is an enveloped virus.

5. The MDBK cell of claim 3, wherein the virus is selected from the group consisting of BRSV, bovine diarrhea virus, bovine rhinotracheitis virus, bovine parvovirus, bovine adenovirus, bovine corona virus and bovine parainfluenza virus.

6. The MDBK cell of claim 3, wherein the virus is a bovine respiratory syncytial virus (BRSV).

7. A cell culture comprising the MDBK cell of claim 1.

8. (canceled)

9. A method for the production of a virus, the method characterized in that it comprises the steps of:
(a) culturing the MDBK cell according to claim 1,
(b) infecting the cell with a virus,
(c) allowing the virus to replicate, and
(d) isolating the virus from the cell culture.

10. A vaccine composition characterized in that it comprises the virus infected cell culture of claim 7 or a virus isolated therefrom and a pharmaceutically acceptable carrier.

11. A method for the preparation of a vaccine composition comprising a virus, the method characterized in that it comprises the steps of:
(a) culturing the MDBK cell according to claim 1,
(b) infecting the cell with a virus,
(c) allowing the virus to replicate,
(d) isolating the virus from the cell culture, and
(e) mixing the virus with a pharmaceutically acceptable carrier.

12. The vaccine composition of claim 10, characterized in that the pharmaceutically acceptable carrier comprises a natural deep-eutectic solvent (NADES) having a water activity of less than about 0.8.

13. The vaccine composition of claim 12, wherein the virus is a live virus or live attenuated virus.

14. The vaccine composition of claim 13, wherein the virus is a bovine respiratory syncytial virus (BRSV).

15. A method of CRISPR-Cas9 mediated gene editing, characterized in that the method comprises the steps of:
(a) providing an MDBK cell and
(b) performing CRISPR-Cas9 mediated functional inactivation of the interferon regulatory factors (IRF) IRF3 and/or IRF7 encoding genes of the MDBK cell.

16. A method for the production of a virus, the method characterized in that it comprises the steps of:
(a) culturing the MDBK cell according to claim 1,
(b) infecting the cell with a virus, and
(c) allowing the virus to replicate.

17. A method for the preparation of a vaccine composition comprising a virus, the method characterized in that it comprises the steps of:
(a) culturing the MDBK cell according to claim 1,
(b) infecting the cell with a virus,
(c) allowing the virus to replicate, and
(d) mixing the cell culture infected with the virus with a pharmaceutically acceptable carrier.

18. The method of claim 17, characterized in that the pharmaceutically acceptable carrier comprises a natural deep-eutectic solvent (NADES) having a water activity of less than about 0.8.
